(19) United States
(12) Patent Application Publication
(10) Pub. No.: US 2022/0346996 A1
(43) Pub. Date: Nov. 3, 2022

(54) DEVICES, SYSTEMS, AND METHODS FOR DUODENAL EXCLUSION AND STOMACH CAPACITY REDUCTION

(71) Applicant: BOSTON SCIENTIFIC SCIMED, INC., MAPLE GROVE, MN (US)

(72) Inventor: John Thomas Favreau, Spencer, MA (US)

(73) Assignee: BOSTON SCIENTIFIC SCIMED, INC., MAPLE GROVE, MN (US)

(21) Appl. No.: 17/730,864

(22) Filed: Apr. 27, 2022

Related U.S. Application Data
(60) Provisional application No. 63/180,853, filed on Apr. 28, 2021.

(51) Int. Cl. A61F 5/00; A61B 17/11; A61F 2/04

(52) U.S. Cl. CPC A61F 5/0079; A61F 5/0089; A61B 17/1114; A61F 2/04; A61B 2017/1139; A61F 2002/045

(57) ABSTRACT

An implantable flow-restricting device, extending along a longitudinal extent which need not be linear, and having a first portion and a second portion with a saddle region therebetween. At least one of the first or second portions may be angled with respect to the saddle region to resist migration of the device. At least one of the first or second portions may be configured to maintain a distance from a region of the anatomical structure in which such portion is positioned. An occluder, such as an expandable occluder, may be associated with the flow-restricting device, such as with one of the first or second portions thereof. The occluder may serve to block passage of material through the anatomical passage in which the saddle region is positioned, prevent migration of the device (e.g., through such passage), and/or increase volume occupied by the device.

### CROSS-REFERENCE TO RELATED APPLICATIONS

This application claims the benefit of priority of U.S. Provisional Application No. 63/180,853, filed Apr. 28, 2021, the entire disclosure of which is hereby incorporated by reference herein for all purposes.

### FIELD

The present disclosure relates generally to the field of implantable medical devices, and related systems and methods, for reducing passage of material through and/or occluding a body passage or lumen. More particularly, the present disclosure relates to devices, systems, and methods for reducing passage of material through and/or occluding a body passage or lumen in the gastrointestinal tract, such as the pylorus. The present disclosure further relates to devices, systems, and methods for reducing passage of material through and/or occluding the pylorus as well as creating a restrictive effect in the stomach.

### BACKGROUND

Treatment methods for various medical conditions, such as obesity, diabetes, or duodenal ulcers, involve bypassing the duodenum or restricting flow of materials through the duodenum. If the treatment requires complete bypass of the duodenum, then occlusion (e.g., full occlusion) of the pylorus may be indicated, and an anastomosis may be created, such as between the stomach and the jejunum. A duodenal exclusion device may be placed in the pyloric sphincter to inhibit or block passage of materials (fluid, liquid, chyme, etc.) from the stomach through the pylorus an into the duodenum. One challenge presented by such devices is to prevent migration of the device distally into the small intestine or proximally into the stomach.

Various medical approaches for treating bariatric or metabolic diseases further include restricting a portion of the stomach and/or reducing the internal volume of the stomach, which has been considered an effective way to reduce food consumption by creating a feeling of satiety. Generally, duodenal exclusion devices do not address reduction of stomach capacity.

It has become increasingly desirable to provide a minimally invasive alternative to existing approaches for treating gastrointestinal or bariatric or metabolic diseases. In particular, it has become increasingly desirable to provide devices, systems, and methods which do not require open surgical procedures, but instead use a transluminal or transcatheter approach, such as an endoscopic procedure (e.g., a natural orifice transluminal endoscopic surgery approach). With the above considerations in mind, a variety of advantageous medical outcomes may be realized by the devices, systems, and/or methods of the present disclosure.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, a flow-restricting device having a longitudinal extent and deployable within an anatomical structure includes a first portion, a second portion, and a saddle region extending between the first portion and the second portion.

In accordance with an aspect of the present disclosure, the first portion of the flow-restricting device is not collinear with the saddle region.

In some embodiments, the first portion of the flow-restricting device has a noncircular cross-sectional shape at least across a region thereof configured to remain spaced apart from a selected anatomical region.

In some embodiments, the flow-restricting device is configured to be positioned with the saddle region extending through a passage between a first anatomical structure in which the first portion is to be positioned and a second anatomical structure in which the second portion is to be positioned. The first portion may have a first region extending along a first region of the first anatomical structure and a second region extending along a second region of the first anatomical structure. The first portion may be angled with respect to the saddle region in a direction away from the second region of the first anatomical structure when positioned in the first anatomical structure. In some embodiments, the first region of the first portion is configured to follow the contour of the first region of the first anatomical structure; and the second region of the first portion is configured to be spaced apart from the second region of the first anatomical structure to distance the first portion from the second region of the first anatomical structure. In some embodiments, the first region is convex on an outer side thereof; and the second region is substantially straight or concave on an outer side thereof. In some embodiments, the cross-sectional area through the first region and the second region of the first portion is smaller than the cross-sectional area of the first anatomical structure in which the device cross-sectional area extends.

In some embodiments, the device is movable between a collapsed configuration and an expanded deployed configuration; and the first portion is sized to resist migration through the passage through which the saddle region extends.

In some embodiments, the flow-restricting device is formed of a plurality of woven strands; the first portion is expanded to have a greater cross-sectional dimension than the second portion; and the first portion is formed of fewer strands than the second portion.

In some embodiments, the flow-restricting device includes an occluder associated with a noncollinear first portion. In some embodiments, the occluder is expandable to increase the volume occupied by the first portion within the first anatomical structure.

In accordance with another aspect of the present disclosure, the flow-restricting device includes an occluder associated with the first portion. In some embodiments, the occluder is selectively expandable. In some embodiments, the first portion is in the form of a cage; and the occluder is positioned within the first portion. In some embodiments, the occluder is positioned within the first portion to restrict passage of materials towards the saddle region. In some embodiments, the occluder is expandable to increase the volume occupied by the first portion.

In accordance with yet another aspect of the present disclosure, the saddle region is configured to be deployed across a pylorus; the first portion is configured to fit within a stomach and is sized and configured to resist distal migration through the pylorus; the second portion is configured to fit within a duodenum and is configured to resist proximal migration through the pylorus; and an expandable occluder is positioned within the first portion to resist distal migration of the flow-restricting device through the pylorus and to occlude passage of materials through the pylorus.

In accordance with yet another aspect of the present disclosure, a system for restricting flow of material through an anatomical structure is provided with a tubular delivery device; a flow-restricting device configured to shift between a collapsed configuration to fit within a lumen within the exterior tubular device, and an expanded deployed configuration when not positioned within the exterior tubular device; an occluder associated with the flow-restricting device; and an interior pusher extending through the tubular delivery device and configured to engage the flow-restricting device. Relative movement of the interior pusher and the exterior tubular delivery device may cause deployment of the flow-restricting device from the tubular delivery device. In some embodiments, the occluder is expandable, and the system further includes an inflation lumen fluidly couplable with the occluder to selectively expand the occluder.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an embodiment of an occlusion device in accordance with various aspects of the present disclosure.

FIG. 2 illustrates a perspective view of an embodiment of an occlusion device formed in accordance with various aspects of the present disclosure and positioned in a schematic representation of a gastrointestinal environment.

FIG. 3 illustrates a perspective view of an embodiment of an occlusion device formed in accordance with various aspects of the present disclosure and positioned in a schematic representation of a gastrointestinal environment with an anastomosis between the stomach and a portion of the small intestines.

FIGS. 4A, 4B, 4C, and 4D illustrate alternate embodiments of cross-sectional views through line IV-IV of FIG. 3.

FIG. 5 illustrates an embodiment of an occlusion device and associated delivery device formed in accordance with various aspects of the present disclosure, with the occlusion device in a collapsed, compact delivery configuration.

FIG. 6 illustrates an embodiment of an occlusion device and associated delivery device formed in accordance with various aspects of the present disclosure, with the occlusion device in a deployed, expanded configuration.

FIG. 7 illustrates a perspective view of an inflation catheter operatively coupled to a balloon of an occlusion device in accordance with various principles of the present disclosure.

FIG. 8A illustrates an example of a cross-sectional view along line VIII-VIII of FIG. 7.

FIG. 8B illustrates a view similar to that of FIG. 8A but with the illustrated example of an inflation catheter decoupled from the illustrated example of a valve.

FIG. 9A and FIG. 9B illustrate elevational views of alternate embodiments of valve seals of a balloon of an occlusion device in accordance with various principles of the present disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and “distal” refers to the direction or location furthest from the user, such as when using the device (e.g., introducing the device into a patient, or during implantation, positioning, or delivery). “Longitudinal” means extending along the longer or larger dimension of an element. “Central” means at least generally bisecting a center point, and a “central axis” means, with respect to an opening, a line that at least generally bisects a center point of the opening, extending longitudinally along the length of the opening when the opening comprises, for example, a tubular element, a channel, a cavity, or a bore.

In accordance with various principles of the present disclosure, a device and associated systems and methods are provided to reduce or prevent passage of material through a body passage or lumen. In some embodiments, the device, system, and methods occlude (fully or substantially fully) passage of material through the body passage or lumen. It will be appreciated that the terms body passage and lumen may be used interchangeably herein without intent to limit, the broad principles of the present disclosure being applicable to various shapes and sizes of body passages/lumens. For the sake of convenience, a device formed in accordance with various principles of the present disclosure may be referenced herein as a flow-restricting device without intent to limit. It will be appreciated that reference to the device as “flow-restricting” includes partially restricting flow of materials (e.g., inhibiting or reducing flow of materials) and fully restricting flow of materials (e.g., preventing or blocking flow of materials). Moreover, it will be appreciated that terms such as restrict, inhibit, occlude, block, prevent, etc. (and conjugations and other grammatical forms thereof) may be used interchangeably herein without intent to limit.

If the body passage includes a passage of a limited extent between different anatomical structures, a flow-restricting device formed in accordance with various principles of the present disclosure may be considered to have a proximal portion configured to fit or to be seated within the proximal anatomical structure, a distal portion configured to fit or to be seated within the distal anatomical structure, and a saddle region therebetween configured to fit or to be seated within the passage extending between the proximal anatomical structure and the distal anatomical structure. It will be appreciated that terms such as fit or seat or position or the like (and conjugations thereof) may be used interchangeably herein without intent to limit unless otherwise indicated. Moreover, it will be appreciated that the term anatomical structure may be used herein to reference any structure or section or region or area in the body such as an organ, vessel, lumen, cavity etc., the term “anatomical structure” being used for the sake of simplicity and without intent to limit.

Various portions of the flow-restricting device may be shaped or configured or provided with various additional features to resist and/or inhibit and/or prevent migration from the deployment site in which the flow-restricting device is positioned for treatment of the patient. It will be appreciated that with reference to migration, terms such as resist, inhibit, prevent, or the like may be used interchangeably herein singly or in combination without intent to limit unless otherwise indicated. In some embodiments, regions of the flow-restricting device may be uncoated or otherwise treated to promote tissue ingrowth to stabilize the device and/or to inhibit migration of the device. In some embodiments, the shape and structure of the flow-restricting device contributes to maintaining the device in the desired deployment location or position. For instance, a portion of the device configured to be positioned in a larger anatomical structure may be deflected or offset or curved or bent or otherwise not aligned with at least an adjacent section of the device configured to be positioned in narrower passage (e.g., a saddle region of the device). Additionally or alternatively, a proximal portion and/or a distal portion of the device may be sized, shaped, and configured to resist migration of the device. For instance, at least one of the proximal portion or the distal portion may have at least a cross-sectional dimension (e.g., a cross-sectional area) larger than the saddle region therebetween to resist passing through the anatomical passage in which the saddle region is positioned. In some embodiments, an occluder may be associated with at least one of the proximal portion or the distal portion and sized, shaped, and configured to resist migration of the flow-restricting device.

In accordance with an aspect of the present disclosure, at least one region of a portion of a flow-restricting device may be contoured to follow or correspond to, such as to substantially match, the contour or shape of the anatomical structure in which such portion of the device is positioned. In some embodiments, such portion of the device may also include at least one region of contoured to avoid a region in the anatomical structure in which such portion of the device is positioned.

In accordance with a separate and independent aspect of the present disclosure which may optionally be applied in conjunction with one or more of the above-described aspects, a separate occluder may be associated with at least one portion of the flow restricting device. The occluder is a plug or wall or other structure that occludes or blocks flow of material. The occluder is deliverable with a flow-restricting device formed in accordance with various principles of the present disclosure. The occluder may be configured to be expandable from a collapsed delivery configuration once deployed with the flow-restricting device. One example of an occluder is an inflatable balloon, though other flow-occluding structures are within the scope and spirit of the present disclosure. The occluder may contribute to or enhance the function of the flow restricting device portion with which it is associated. For instance, the occluder may contribute to occluding flow of materials through the flow-restricting device, such as through a saddle region thereof. Additionally or alternatively, an occluder may assist in resisting migration of the flow-restricting device. Additionally or alternatively, the occluder may be configured to occupy volume and thus may also serve to reduce the functional volume of the anatomical structure occupied by the flow restricting device. In some embodiments, the volume of the occluder is adjustable, such as during placement of the device and/or during the course of treatment to adjust the rate of flow of materials past the portion of the device with the occluder (such as through the passage through which the device is positioned) and/or to adjust the volume occupied by the occluder. In some embodiments, the occluder is maintained in a desired position in the anatomical structure by its position within a portion of the flow-restricting device (e.g., “captured” or otherwise held or restrained within a portion of the flow-restricting device). As such, an occluder which may be used separately from a flow-restricting device formed in accordance with various principles of the present disclosure may benefit from features of the flow-restricting device, such as designs or features or structures provided to resist migration of the device from the desired deployment position in the patient.

An example of an environment in which devices, systems, and methods of the present disclosure may be used is the gastrointestinal system. Because the pylorus is a narrowed structure at the distal region of the stomach, in some embodiments, the gastric section of a flow-restricting device may be significantly larger than the saddle region configured for positioning within the pylorus to resist distal migration through the pylorus. For instance, the stomach may be approximately 10 cm in diameter at its widest part, the pylorus may be approximately 2 cm or less (typically about 1 cm and can contract to close to 0 cm) in diameter, and the portion of the duodenum adjacent the pylorus may be about 2-3 cm in diameter. Accordingly, the gastric section of a flow-restricting device formed in accordance with various principles of the present disclosure may have a diameter of approximately 2-2.5 times the diameter of the duodenal section of the device. The diameter of the saddle region (extending through the pylorus) of a flow-restricting device formed in accordance with various principles of the present disclosure generally is not critical to the present disclosure, and may be smaller than the diameter of the pylorus (e.g., as small as 1-2 mm in diameter, and may be fully closed), and thus very small relative to the other sections of the device without affecting other aspects of the present disclosure.

Devices, systems, and methods in accordance with various principles of the present disclosure may be used to reduce and/or slow the rate of passage of materials through the pylorus and/or to occlude/exclude the pylorus from the stomach, such as for gastric procedures such as bariatric procedures, or to treat other gastrointestinal conditions. The gastric portion and/or the saddle region of a flow-restricting device formed in accordance with various principles of the present disclosure may be structured to occlude (partially or fully/completely) flow therethrough and, consequently, flow of material through the pylorus.

In some instances, it may be desirable to bypass the duodenum, such as by occluding (completely or almost completely) the pylorus and by creating an anastomosis to join the stomach with the jejunum. For example, occluding duodenal access and redirecting food, liquid, and other nutrients through an alternative path, delaying interaction of the stomach content with digestive enzymes until further down the small intestines (and optionally effectively bypassing the pancreas), may reduce obesity and/or a patient's risk of type-2 diabetes. When a flow-restricting device formed in accordance with various principles of the present disclosure is positioned across a pylorus, the gastric portion of the device may be larger than the duodenal portion of the device (such as in view of the generally larger size of the stomach relative to the duodenum). In accordance with one aspect of the present disclosure, a device not only occludes an anatomical passage, such as a pylorus, but also is configured so as not to interfere with or impede or otherwise interact with an anastomosis bypassing the anatomical passage. The gastric section of a flow-restricting device formed in accordance with various principles of the present disclosure may be deflected or otherwise contoured to remain spaced away from or to extend around or otherwise not to engage or interfere with or block the anastomosis.

A flow-restricting device formed in accordance with various principles of the present disclosure may also be used in bariatric treatments involving increasing the feeling of satiety in the patient, with the intent to reduce the desire to eat, with consequent reduction in caloric intake. Various approaches to increasing the feeling of satiety include increasing the time food remains in the stomach and/or reducing or slowing the rate of gastric emptying (the flow of material, such as chyme, from the stomach to the duodenum). The above-described pyloric occlusion devices and methods may be advantageously used for such additional purpose. Another approach to increasing the feeling of satiety is to implant a device which occupies volume in the stomach. A flow-restricting device with an occluder formed in accordance with principles of the present disclosure as described above may be positioned across a pylorus, with an occluder associated with a proximal portion of the device positioned within the stomach. As such, the occluder fills a portion of the stomach to reduce the apparent volume of the stomach, with consequent inducement of a feeling of fullness or satiety which may lead to reduction of food intake and associated weight loss. In some embodiments, the proximal portion of the flow-restricting device may form a frame or cage around the occluder, capturing the occluder to maintain the occluder in a desired position within the stomach. Such configuration may be particularly advantageous when this form of treatment is used in conjunction with a gastric bypass. The flow-restricting device may hold the occluder away from the gastrojejunal anastomosis (such as described above with respect to a configuration of a flow-restricting device diverted or deflected away from the anastomosis). Such configuration may also contribute to the anti-migration effect of the enlarged proximal portion of the flow-restricting device, further reducing the likelihood of the proximal portion migrating distally through the pylorus.

In some embodiments, it may be desirable to adjust the flow-restricting device and/or a portion thereof, such an occluder, while still implanted in the patient. For instance, the volume of an expandable occluder associated with a flow-restricting device formed in accordance with various principles of the present disclosure may be adjustable. Positioning of the occluder within a portion of the flow-restricting device may advantageously maintain the occluder in an accessible position for adjustment thereof while the flow-restricting device remains seated in the desired deployment site. Additionally or alternatively, it may be desirable to remove a flow-restricting device formed in accordance with various principles of the present disclosure. For instance, a pyloric closure device may be removable or adjustable.

It will be appreciated that devices, systems, and methods as disclosed herein may be used in endoscopic, laparoscopic, and/or open surgical procedure. Preferably, a medical professional may be able to deliver and/or to remove the device endoscopically. Advantageously, devices and systems disclosed herein may be used in minimally invasive procedures such as natural orifice transluminal endoscopic surgery (NOTES).

An implantable treatment device formed in accordance with principles of the present disclosure may be provided as part of a treatment system. For instance, a delivery device may be configured as an elongated flexible delivery device capable of navigating through internal passages within the patient to avoid open surgery. The flow-restricting device may be delivered in a collapsed configuration within the delivery device to the deployment site. An additional deployment device, such as a pusher, may be provided to facilitate deployment of the flow-restricting device from the delivery device (e.g., by ejecting the flow-restricting device from its position with the delivery device). Once deployed, the flow-restricting device moves or shifts into an expanded deployed configuration. If an expandable occluder is provided, then the treatment system may further include an inflation lumen couplable to the occluder. The inflatable occluder may be configured to be couplable with an inflation lumen after deployment, such as for adjustment at later time during the course of treatment after initial deployment.

A method of treatment utilizing an implantable device involves delivery of the device to the deployment site, and deployment of the device, such as by withdrawing the delivery device proximally, or by utilizing a pusher to distally move the device out of the delivery device, or a combination thereof. Optionally, the method of treatment involves providing, and optionally expanding, an occluder to effect a desired aspect of the treatment protocol. The device may be adjusted at any time after deployment and during the course of treatment. The device may be removed if the treatment has been deemed successful and use of the flow-restricting device no longer indicated.

Although devices, systems, and methods are described herein with respect to a gastrointestinal system, it may be understood that embodiments of devices, systems, and methods in accordance with the present disclosure may be advantageous for use in other procedures and/or anatomical structures. Reference may be made herein to an implantable device, a device, a stent, or the like, such terms usable interchangeably herein without intent to limit.

Various embodiments of flow-restricting devices will now be described with reference to examples illustrated in the accompanying drawings. Reference in this specification to “one embodiment,” “an embodiment,” “some embodiments”, “other embodiments”, etc. indicates that one or more particular features, structures, and/or characteristics in accordance with principles of the present disclosure may be included in connection with the embodiment. However, such references do not necessarily mean that all embodiments include the particular features, structures, and/or characteristics, or that an embodiment includes all features, structures, and/or characteristics. Some embodiments may include one or more such features, structures, and/or characteristics, in various combinations thereof. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. When particular features, structures, and/or characteristics are described in connection with one embodiment, it should be understood that such features, structures, and/or characteristics may also be used in connection with other embodiments whether or not explicitly described, unless clearly stated to the contrary. It should further be understood that such features, structures, and/or characteristics may be used or present singly or in various combinations with one another to create alternative embodiments which are considered part of the present disclosure, as it would be too cumbersome to describe all of the numerous possible combinations and subcombinations of features, structures, and/or characteristics. Moreover, various features, structures, and/or characteristics are described which may be exhibited by some embodiments and not by others. Similarly, various features, structures, and/or characteristics or requirements are described which may be features, structures, and/or characteristics or requirements for some embodiments but may not be features, structures, and/or characteristics or requirements for other embodiments. Therefore, the present invention is not limited to only the embodiments specifically described herein.

Turning now to the drawings, an example of a flow-restricting device 100 formed in accordance with various principles of the present disclosure is illustrated in FIG. 1 having a proximal portion 110, a distal portion 120, and a saddle region 130 therebetween. As may be appreciated with reference to FIG. 1, the cross-sectional dimensions of the flow-restricting device 100 vary along its longitudinal extent L (extending in a direction between the proximal end 101 and the distal end 103 of the flow-restricting device 100), with the saddle region 130 being narrower than the proximal portion 110 and the distal portion 120. At least a portion of the flow-restricting device 100 is configured to restrict or prevent flow of materials therethrough or through a passage through which the flow-restricting device 100 is positioned. Various structures or features known or heretofore known in the art may be used to result in the desired flow-restricting configuration of the flow-restricting device 100. For instance, in embodiments with a saddle region 130 narrower than a proximal portion 110 and a distal portion 120 of the flow-restricting device 100, the saddle region 130 may have a generally hollow configuration and may be twisted or rotated to create a kink or closure element or otherwise to increase the density of the material forming the walls of the saddle region 130 to occlude flow of material therethrough. In other embodiments, the saddle region 130 may be generally solid. In some embodiments, the saddle region 130 is so narrow as to be negligible in width relative to the proximal portion 110 and the distal portion 120.

In the illustrated embodiment, the configuration of the proximal portion 110 is different from the configuration of the distal portion 120, although other configurations are within the scope of the present disclosure. Additionally, the proximal portion 110 and the distal portion 120 of the illustrated embodiment have a generally larger cross-sectional dimensions (e.g., width or area) than a cross-sectional dimension (e.g., a corresponding cross-sectional dimension, such as widths in generally the same direction) of the saddle region 130. In such embodiment, the saddle region 130 may be positioned across a passage between a proximal anatomical structure in which the proximal portion 110 of the flow-restricting device 100 is positioned, and a distal anatomical structure in which the distal portion 120 of the flow-restricting device 100 is positioned. The proximal and distal anatomical structures may have different cross-sectional shapes or dimensions or volumes. Accordingly, the proximal portion 110 and the distal portion 120 may not be symmetrical and/or may not have the same dimensions (e.g., cross-sectional or longitudinal dimensions). At least one, and preferably both of the proximal portion 110 and the distal portion 120 are shaped and configured and/or provided with one or more features to inhibit migration of the flow-restricting device 100 through the body passage in which the saddle region 130 is positioned or deployed. It will be appreciated that terms such as positioned, deployed, etc. (including conjugations thereof) may be used interchangeably herein without intent to limit.

Various features of a flow-restricting device 100 formed in accordance with various principles of the present disclosure are described herein with reference to the figures and examples of environments in which a flow-restricting device 100 formed in accordance with principles of the present disclosure may be used. However, it will be appreciated that the principles of the present disclosure have broader applications than the illustrated examples and the descriptions thereof.

The flow-restricting device 100 may be at least partially formed from a plurality of strands or wires or filaments which may be braided or woven or twisted or wrapped or intertwined or knitted or looped (e.g., bobbinet-style) or knotted or otherwise formed into a self-supporting structure. Alternatively, the flow-restricting device 100 may be at least partially formed from a laser-cut tube or scaffold or bonded elongated elements, or a combination of a self-expanding metal stent and laser-cut tube or scaffold, or another self-supporting structure. Such structure may be referenced as a stent or framework or scaffold without intent to limit. The flow-restricting device 100 may be at least partially formed of a biocompatible metal or a polymeric material or an alloy. In some embodiments, the material is a shape-memory or heat formable material, such as a nickel-titanium alloy (e.g., nitinol). In accordance with various principles of the present disclosure, the flow-restricting device 100 may be sized and configured for transluminal or transcatheter or endoscopic delivery. As such, in accordance with an aspect of the present disclosure, the flow-restricting device 100 may be collapsed or otherwise reduced in cross-sectional dimension to fit through a tubular delivery device used in minimally-invasive procedures (in contrast with an open surgical procedure). The flow-restricting device 100 may expand once deployed. For instance, the flow-restricting device 100 may be formed to be self-expanding (and, in such case, advantageously formed of a shape memory material which expands the device once no longer held or constrained within a delivery device), or may be expanded with the assistance of another expandable device, such as an expandable balloon. To allow the option of removal from the deployment site, the flow-restricting device 100 may be formed to be selectively collapsible from its expanded deployed configuration (illustrated in FIG. 1). The flow-restricting device 100 preferably is configured such that the cross-sectional dimension Xₚ of the proximal portion 110 (in an expanded configuration) and the cross-sectional dimension X_{D} of the distal portion 120 (in an expanded configuration) as well as the mechanism for collapsing the flow-restricting device 100 inhibit or prevent collapse of the flow-restricting device 100 and distal or proximal migration of the flow-restricting device 100. Various known sheaths or coatings, such as polymeric or elastomeric or silicone or lubricious coatings, or heretofore known coatings, may be applied to selected regions of the flow-restricting device 100 contribute to the mechanics of the device, such as to impart structural stability, and/or to inhibit tissue ingrowth. Selected regions of the flow-restricting device 100 may be remain uncoated to permit tissue ingrowth to resist migration of the flow-restricting device 100), with coated regions having structural stability to facilitate sufficient immobility to allow time for tissue ingrowth into the uncoated regions.

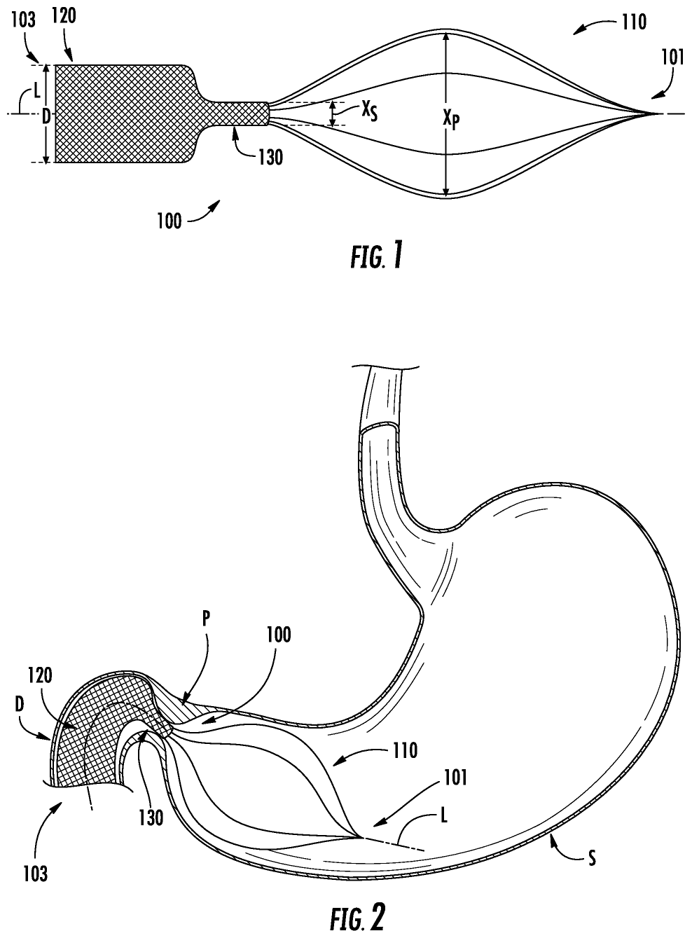

An example of a flow-restricting device 100 formed in accordance with principles of the present disclosure, such as illustrated in FIG. 1, is illustrated in FIG. 2 in place in a schematic representation of an example of a gastrointestinal (“GI”) tract within a human body. In the non-limiting example illustrated in FIG. 2, the flow-restricting device 100 is positioned across a pylorus P, with the proximal portion 110 positioned within a stomach S, the distal portion 120 positioned within a duodenum D, and the saddle region 130 extending between the proximal portion 110 and the distal portion 120 and positioned within the pylorus P.

In the illustrated examples of embodiments of a flow-restricting device 100, the distal portion 120 has a cross-sectional dimension X_{D} larger than a corresponding cross-sectional dimension Xs (e.g., area or a linear dimension in the same general direction) of the saddle region 130. As such, when the flow-restricting device 100 is deployed, the distal portion 120 fits securely within the duodenum D and cannot readily unintentionally pass or migrate proximally through the pylorus P. In some embodiments, additional anti-migration features may be provided in connection with the distal portion 120. For instance, a region or area of the distal portion 120, such as a proximally-facing section seated against the pylorus P, may be uncoated to allow tissue growth into such region. Other regions of the distal portion 120 may be coated, such as with biocompatible coatings known or heretofore known in the art, to inhibit tissue ingrowth and/or to enforce the structure of the distal portion 120 (e.g., to resist collapse thereof and proximal migration through the pylorus P). The distalmost end of the distal portion 120 at the distal end 103 of the flow-restricting device 100 may be coated, such as to coat the tips or ends of wires forming the flow-restricting device 100.

The distal portion 120 may have a longer extent than illustrated, extending further into the duodenum D. Alternatively, a generally flexible sleeve portion (e.g., formed of a knitted or braided material such as known or heretofore in the art) may extend distally from the distal portion 120 of the flow-restricting device 100 to exclude portions of the duodenum should materials pass through the flow-restricting device 100 and the pylorus P (such as initially after deployment of the flow-restricting device 100). The flow-restricting device 100 may be made of Nitinol, cobalt chromium, stainless steel, or other biocompatible metals known or heretofore known in the art, or any of a variety of plastics such as polytetrafluoroethylene (PTFE), polyether block amides (e.g., PEBAX®), polyether ether ketone (PEEK), high density polyethylene (HDPE), polyurethane, or other biocompatible plastics known or heretofore known in the art, or other suitable materials known or heretofore known in the art. The flow-restricting device 100 may be coated with silicone or another elastomeric material or with PTFE or another thin polymer material.

In accordance with various principles of the present disclosure, a flow-restricting device 100 configured for positioning across a pylorus P (or across another anatomical passage between anatomical regions of different sizes), has a proximal portion 110 with a significantly larger cross-sectional dimension Xₚ than the corresponding cross-sectional dimension X_{D} of the distal portion 120. The proximal portion 110 may be formed such that a cross-sectional dimension Xₚ of the proximal portion 110 inhibits or prevents passing or migration of the flow-restricting device 100 distally through the pylorus P. The proximal portion 110 may have additional features further inhibiting or preventing distal migration of the flow-restricting device 100 through the pylorus P, as will now be described.

The embodiment of a flow-restricting device 100 illustrated in FIG. 1 and FIG. 2 is formed of a plurality of wires or strands or filaments (referenced herein as wires for the sake of convenience and without intent to limit). Wires may extend continuously from the proximal portion 110 to the distal portion 120 of the flow-restricting device 100. Alternatively or additionally, separate wires may be used for the proximal portion 110 and the distal portion 120 of the flow-restricting device 100, the wires being welded, glued, or otherwise attached to one another or otherwise coupled (e.g., to a separate section along the saddle region 130) to form the flow-restricting device 100. The spacing among the wires of the larger (at least in cross-section) proximal portion 110 of the flow-restricting device 100 may be greater than the spacing among the wires of the smaller (at least in cross-section) distal portion 120 of the flow-restricting device 100. Such formation allows the proximal portion 110 to have a sufficiently small cross-sectional dimension in a collapsed configuration for delivery, yet a sufficiently large enough cross-sectional dimension to remain seated in a stomach S (or other body cavity or anatomical structure at an end of a body passage through which the flow-restricting device 100 is positioned). If the spacing among wires of the proximal portion 110 is the same as the spacing among wires of the distal portion 120, the collapsed proximal portion 110 may have too many wires to have a sufficiently small enough cross-sectional diameter to fit in a tubular delivery device that can be passed through a body lumen (e.g., the mouth and esophagus, in contrast with cutting open the patient). Alternatively or additionally, the diameter of the wires in the proximal portion 110 of the flow-restricting device 100 may be different from (such as larger than) the diameter of the wires in the distal portion 120 of the flow-restricting device 100. For instance a continuous wire extending through both the proximal portion 110 and the distal portion 120 may have different ground diameters along its length (for positioning in different portions 110, 120 of the flow-restricting device 100). Alternatively or additionally, different wires may be used in each section 110, 120 of the flow-restricting device 100.

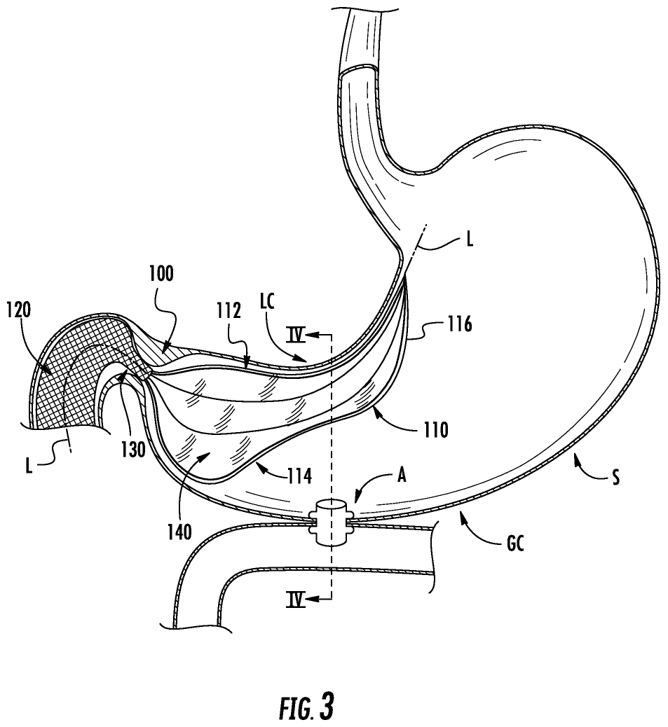

In accordance with an aspect of the present disclosure, as illustrated in FIG. 3, the longitudinal extent L of a flow-restricting device 100 formed in accordance with principles of the present disclosure need not be linear (e.g., straight). Because various anatomical passages are not precisely straight, the longitudinal extent L of a flow-restricting device 100 formed in accordance with various aspects of the present disclosure may follow the nonlinear configuration of the anatomical passage in which the flow-restricting device 100 is to be positioned. For instance, at least the saddle region 130 may be somewhat curved. In accordance with a further aspect of the present disclosure, if at least one portion of the flow-restricting device 100 is positioned in an enlarged anatomical region, such as a body cavity like the stomach S, such portion of the flow-restricting device 100 may be offset or otherwise not aligned with the saddle region 130. More particularly, the saddle region 130 may be considered to have a major or longitudinal axis extending along the longitudinal extent L of the flow-restricting device 100 and in a direction along which the saddle region 130 extends through the pylorus (generally along the largest/longest dimension of the saddle region 130). The proximal portion 110 and/or the distal portion 120 of the flow-restricting device 100 may extend at an angle to/be askew from the major or longitudinal axis of the saddle region 130. Such configuration may reduce the ability of such proximal portion 110 or distal portion 120 to migrate through the body passage through which the saddle region 130 extends. In particular, if such portion of the flow-restricting device 100 is enlarged as well as angled with respect to or otherwise not aligned with the saddle region 130, then such portion has greater difficulty migrating through the body passage then if such portion were collinear with (e.g., aligned with and coextensive and substantially parallel to) the saddle region 130.

In the embodiment illustrated in FIG. 3, in which the flow-restricting device 100 is configured for positioning across a pylorus P, the angle of the proximal portion 110 with respect to the saddle region 130 may also serve to maintain the proximal portion 110 at a distance from an anastomosis A (such as a gastrojejunostomy). In particular, the proximal portion 110 may be angled with respect to the saddle region 130 in a direction away from the anastomosis A. In some embodiments, the outer contour of at least a portion of the proximal portion 110 of the flow-restricting device 100 may generally follow (e.g., generally match or correspond with) the contour of the stomach S. In view of the typical natural contour of the stomach S, if a first region 112 of the flow-restricting device 100 follows the lesser curvature LC of the stomach S (the upper region of the stomach having a generally concave contour between the esophageal sphincter and the pyloric sphincter), such first region 112 may be considered to be generally concave along the longitudinal extent L of the flow-restricting device 100. If such first region 112 is rotated towards the anastomosis A, the concave curvature of such region of the proximal portion 110 generally would maintain a space between the wall of the proximal portion 110 and the anastomosis A. Also in view of the typical natural contour of the stomach S, the concave region 112 and incline thereof with respect to the saddle region 130 may contribute to inhibiting or preventing the flow-restricting device 100 from rotating within the pylorus. In particular, because of the generally convex curvature of the greater curvature GC of the stomach S, the corresponding concave curvature of the interior of such portion of the stomach S extends inwardly greater than the opposite side along the lesser curvature LC of the stomach S, leaving less or no room for the concave region 112 of the flow-restricting device 100 to move into such region in the stomach S. At least the proximal portion 110 of the flow-restricting device 100 may be sufficiently resistant to bending to further inhibit bending of the proximal portion 110 to rotate out of position upon encountering the interior side of the stomach S along the greater curvature GC. It will be appreciated that radiopaque markings or other markings or other technologies (e.g., platinum cored wires) visible with fluoroscopy or other imaging technology as known to those of ordinary skill in the art may be used to position the flow-restricting device 100 with the proximal portion 110 in the desired initial orientation.

Alternatively or additionally, in accordance with various aspects of the present disclosure, a flow-restricting device 100 formed in accordance with various principles of the present disclosure may be configured with other contours or contoured sections or regions to avoid or otherwise to maintain a distance between a wall of the flow-restricting device 100 and another region of the anatomy. Such contours or contoured sections or regions may be shaped differently from surrounding regions of the flow-restricting device 100 (e.g., walls of the flow-restricting device 100). For instance, at least a portion of the longitudinal extent L of the proximal portion 110 of the flow-restricting device 100 may have a cross-sectional shape with a first region 112 configured to follow the contour of a first area or region of the anatomical section in which the proximal portion 110 is positioned (e.g., a convex outer cross-sectional shape), and a second region 114 configured to be spaced apart from a second area or region of the anatomical section in which the proximal portion 110 is positioned. The second region 114 may be substantially straight (at least relative to the first region 112) or concave or otherwise configured to leave space between the second region 114 and an anatomical region to be avoided along which the flow-restricting device 100 is positioned. In some embodiments, the outer cross-sectional of the flow-restricting device 100 extending through the second region 114 is noncircular.

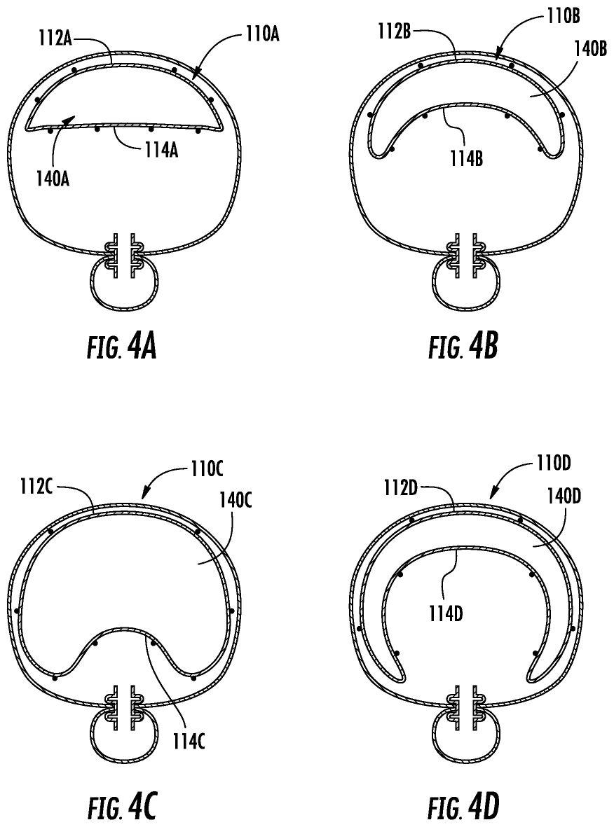

In the embodiment illustrated in FIG. 3, the flow-restricting device 100 is positioned across a pylorus P of a stomach S in which an anastomosis A (e.g., a gastrojejunostomy) has been formed. As may be appreciated with reference to FIG. 3, the proximal portion 110 of the flow-restricting device 100 has a first region 112 facing away from the anastomosis A, and a second region 114 contoured or shaped to be spaced apart from the anastomosis A. The first region 112 may be contoured or shaped to follow the contour of the inner wall of the stomach S, as illustrated. The stomach S has a generally circular cross-sectional shape with the interior wall generally concave along a perimeter at a given position along the longitudinal extent of the stomach between the esophageal sphincter and the pyloric sphincter. Thus, the first region 112 of the proximal portion 110 of the flow-restricting device 100 illustrated in FIG. 3 has a generally concave curvature along the longitudinal extent L of the flow-restricting device 100 (to follow the interior shape of the lesser curvature LC of the stomach S) yet has a generally convex outer cross-sectional shape to follow the concave cross-sectional shape of the stomach S. The second region 114 of the proximal portion 110 of a flow-restricting device 100 is configured to avoid a region of a stomach S in accordance with various principles of the present disclosure. In some embodiments, the second region 114 may have an outer cross-sectional shape which is not convex. Various embodiments of cross-sectional shapes of a flow-restricting device 100, such as along line IV-IV of FIG. 3, are shown in FIGS. 4A, 4B, 4C, 4D. As may be appreciated, the cross-sectional area of the proximal portion 110 of the flow-restricting device 100 in the area of the anatomical region to be avoided (e.g., an anastomosis A) may not occupy the entire cross-sectional area of the anatomical region in which it is positioned. For instance, the proximal portion 110 may occupy an area excluding the anatomical region to be avoided. The cross-sectional area of the proximal portion 110 may be half or less than half of the cross-sectional area of the anatomical region to be avoided (e.g., a cross-section taken along a plane in which the area to be avoided lies).

In the example illustrated in FIG. 4A, a proximal portion 110A of a flow-restricting device 100, configured as illustrated in FIG. 3 to avoid a region of a stomach S, has a cross-sectional shape along line IV-IV with a second region 114A spaced apart from the region of the stomach S to be avoided (such as a gastrojejunal anastomosis A). The second region 114A of this embodiment has an outer cross-sectional shape which is generally at least linear. As may be appreciated with reference to FIG. 4B, illustrating another embodiment of a cross-section along line IV-IV of the flow-restricting device 100 of FIG. 3, the second region 114B may have an outer cross-sectional shape which is generally convex to remain spaced apart from the anatomical region to be avoided. As may be appreciated with reference to the embodiments of FIG. 4A and FIG. 4B, the cross-sectional area of the proximal portion 110A, 110B, respectively, of a flow-restricting device 100 as in FIG. 3 may occupy an area excluding the anatomical region to be avoided, and may occupy half or less than half of the cross-sectional area along a plane intersecting the region to be avoided. It will be appreciated that the second region 114A, 114B need not be generally linear or even concave if spaced far enough from the anatomical region to be avoided.

In other embodiments, such as illustrated in FIG. 4C and FIG. 4D, the proximal portion 110C, 110D may extend a greater distance across the cross-section of the flow-restricting device 100 intersecting a region to be avoided, and may have a second region 114C, 114D more distinctly shaped to avoid a region to be avoided. For instance, as illustrated in FIG. 4C, the cross-sectional area of a proximal portion 110C may occupy more than half of the cross-sectional area of the anatomical region if the outer cross-sectional shape of the second region 114C is concave and sufficiently distanced from the region to be avoided, such as illustrated in FIG. 4C. As may be appreciated, the first region 112 has a substantially convex outer cross-sectional shape, as does the majority of the outer perimeter of the proximal portion 110 along line IV-IV. In another embodiment, illustrated in FIG. 4D, the cross-sectional area of a proximal portion 110D may occupy less than half of the cross-sectional area of the anatomical structure intersecting the anatomical region to be avoided, yet may still extend across more than half the cross-section if the outer cross-sectional shape of the second region 114D is sufficiently concave. As illustrated, the outer contour of the second region 114D may substantially follow the outer contour of the first region 112D, such that the walls of these portions may remain substantially equidistant from each other, the outer surface of the second region 114D thereby remaining sufficiently spaced away from the anatomical region to be avoided. In view of the above discussions, if the proximal portion 110C, 110D of the flow-restricting device 100 is curved to follow the lesser curvature of the stomach S, then rotation of the proximal portion 110C, 110D is generally inhibited by such curvature encountering the generally concave inner contour the stomach S in the region of the greater curvature of the stomach S.

It will be appreciated that further variations in cross-sectional shapes along line IV-IV of the proximal portion 110 of a flow-restricting device 100 such as illustrated in FIG. 3 are within the scope of the present disclosure. It is noted that in the embodiment illustrated in FIG. 3 the proximal portion 110 may have a first region 112 extending longitudinally with a concave curvature (e.g., be concave along the longitudinal extent of the flow-restricting device 100) as well as a first region 114 having a cross-sectional shape configured to be spaced away from an anatomical region to be avoided. However, in other embodiments, a proximal portion 110 may have only one of such characteristics or contours.

In accordance with a separate and independent aspect of the present disclosure, an occluder 140 may be associated with the flow-restricting device 100, such as with the proximal portion 110, as illustrated in FIG. 3. It will be appreciated that the occluder 140 may be provided with any combination (including none) of the above-described aspects, such as a portion of a flow-restricting device 100 being angled with respect to the saddle region 130, and/or a portion of the flow-restricting device 100 being configured to avoid a region of the anatomical structure in which such portion of the flow-restricting device 100 is positioned. In the embodiment illustrated in FIG. 3, the occluder 140 is associated with the proximal portion 110 of the flow-restricting device 100. As may be appreciated, the occluder 140 may contribute to the volume and/or structural integrity of the proximal portion 110 to resist collapse and to resist distal migration through the pylorus P. The occluder 140 may be positioned to occlude passage of material through the body passage through which the flow-restricting device 100 (e.g., the saddle region 130 thereof) is positioned. Additionally or alternatively, the occluder 140 may occupy volume within the anatomical region in which it is positioned to achieve various treatment effects. The occluder 140 preferably is adjustable in size and/or position with respect to the proximal portion 110. For instance, the occluder 140 can be selectively expandable to different extents, such as to vary the degree to which the occluder 140 blocks passage of material and/or to vary the volume occupied by the occluder 140.

As discussed above, the embodiment of a flow-restricting device 100 illustrated in FIG. 3 is positioned across a pylorus P to restrict flow of material through the pylorus P. In some embodiments, the saddle region 130 has a lumen therethrough which is narrower than the passage through which the saddle region 130 is positioned to restrict the flow of materials through the lumen. In some embodiments, the lumen through the saddle region 130 may be substantially fully closed to occlude (e.g., fully obstruct) flow of materials therethrough. In other embodiments, the lumen through the saddle region 130 only partially reduces flow therethrough and/or the saddle region 130 only partially reduces flow through the pylorus P, and the proximal portion 110 may include further features restricting flow of material. The occluder 140 may contribute to restrict flow of material through the pylorus P by impeding flow of material into the pylorus P. As discussed above, in some instances, it may be desirable to reduce the volume of the stomach S in which the flow-restricting device 100 is positioned, such as in bariatric procedures. The occluder 140 occupies additional volume in the stomach S for such mode of treatment.

In accordance with an aspect of the present disclosure, the occluder 140 may be selectively expandable to achieve and to impart an indicated amount of occlusion and/or to occupy an indicated of volume. For instance, the occluder 140 may be fully expanded to fully occlude entry into the passage (e.g., pylorus P) through which the flow-restricting device 100 (e.g., the saddle region 130 of the flow-restricting device 100) is positioned, or may be partially expanded to reduce but not fully occlude the passage. If the occluder 140 is used in a bariatric course of treatment, then the volume of the occluder 140 may be adjusted during the course of treatment if the patient is losing weight successfully (and may not need as a great a reduction in stomach volume created by the inflated occluder 140), or if increased weight loss is indicated (and increased filling of the stomach volume is desired by increasing the volume of the occluder 140).

In accordance with various principles of the present disclosure, the occluder 140 may be formed from an elastic material permitting inflation of the occluder 140. For instance, the occluder 140 may be a balloon selectively fillable with air or saline or another appropriate substance (e.g., gas or fluid) to occupy all or just a portion of the interior volume of the proximal portion 110 of the flow-restricting device 100. The material may be compliant or non-compliant, generally depending on the desired use of the occluder 140. In some embodiments, the occluder 140 is formed of a compliant material facilitating collapse thereof into a compact configuration for transcatheter or transluminal delivery as described in further detail below, and inflation to varying extents (as medically indicated) when delivered to the deployment site. It will be appreciated that the occluder 140 may have an outer cross-sectional shape matching the inner cross-sectional shape of the portion 110, 120 of the flow-restricting device 100 in which the occluder 140 is positioned, such as any of the shapes illustrated in FIG. 4A, 4B, 4C, or 4D. Alternatively, the occluder 140 may have an outer cross-sectional shape different from the inner cross-sectional shape of the portion 110, 120 of the flow-restricting device 100 in which the occluder 140 is positioned, such as a shape (e.g., circular or oval) occupying less than the total cross-sectional area of the portion 110, 120.

The occluder 140 may be positioned in one or both of the proximal portion 110 and the distal portion 120 of the flow-restricting device 100. In the illustrated embodiment of FIG. 3, the occluder 140 is positioned in just the proximal portion 110 and within a stomach S. It will be appreciated that the structure of the portion of the flow-restricting device 100 in which the occluder 140 is positioned may form a cage or frame which holds or otherwise maintains the occluder 140 in a desired position or location at the deployment site. For instance, if it is desirable to maintain the flow-restricting device 100 away from a particular region of the anatomical structure in which the flow-restricting device 100 is deployed, the proximal portion 110 may be configured as described above to avoid such region as well as to hold the occluder 140 away from such region. Additionally or alternatively, the proximal portion 110 may hold the occluder 140 in a general region of the anatomical structure in which the flow-restricting device 100 is positioned for other reasons. For instance, if it is desirable to adjust the volume of the occluder 140 (e.g., during the course of treatment), the portion of the flow-restricting device 100 in which the occluder 140 is positioned may generally maintain the occluder 140 sufficiently stable for access thereto. The occluder 140 is not subject to drift significantly if contained within a portion of the flow-restricting device 100, thereby facilitating access thereto, such as for adjustment thereof.

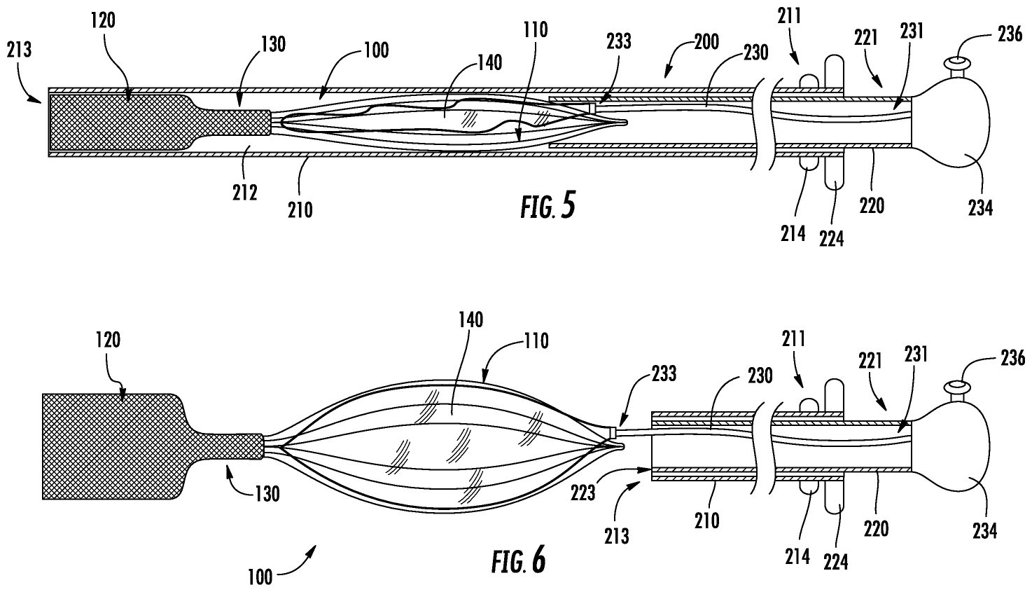

Delivery and deployment of a flow-restricting device 100 formed in accordance with various principles of the present disclosure will now be described with reference to an example of a delivery device 200 illustrated in FIGS. 5 and 6. As noted above, the flow-restricting device 100 (including an occluder 140 included therewith) may be configured to be collapsed for transcatheter or transluminal delivery. In the embodiment illustrated in FIG. 5, the delivery device 200 includes a flexible tubular element 210 (such as a catheter or other flexible elongate member with a lumen 212 therethrough) capable of readily fitting within and being transported through the lumen of a delivery device extended through the body (e.g., through a passage within the body rather than through an opening surgically created in the body). The flow-restricting device 100 is illustrated in a collapsed or compact configuration within the lumen 212 of the flexible tubular element 210. In the illustrated embodiment, an occluder 140 is positioned within the proximal portion 110 of the flow-restricting device 100. Accordingly, the overall cross-sectional area of the proximal portion 110 with the occluder 140 therein may be larger than the overall cross-sectional area of either of the distal portion 120 or the saddle region 130. Nonetheless, the overall cross-sectional area of the proximal portion 110 with the occluder 140 therein is sized to fit within and be transported through the lumen 212 of the delivery device 200.

The delivery device 200 with the flow-restricting device 100 collapsed therein is preferably sufficiently flexible to be navigated through a natural body passage or lumen of a patient (e.g., without surgical intervention such as cutting open the patient to facilitate entry and delivery of the delivery device 200) to a delivery/deployment site. Once at the deployment site, an inner tube 220 (slidably positioned within the exterior or outer flexible tubular element 210) may be used as a pusher to assist with pushing the flow-restricting device 100 out of the flexible tubular element 210. The flexible tubular element 210 and the inner tube 220 extend proximally to a position outside the patient for control thereof by a medical professional. A lumen or other passage may be provided (e.g., provided within the delivery device 200) for a guidewire that may be used to assist with placement and/or deployment of the flow-restricting device 100 in its final position. In some embodiments, as illustrated in FIGS. 5 and 6, the proximal end 211 of the flexible tubular element 210 ends at a control handle 214. Similarly, in some embodiments, as illustrated in FIGS. 5 and 6, the proximal end 221 of the inner tube 220 ends at a control handle 224. The control handles 214, 224 may be provided on a common control handle assembly, or may be separately formed. Preferably, the control handles 214, 224 are configured to separately control movement of the flexible tubular element 210 and the inner tube 220 with respect to each other, as will be appreciated with reference to the following description of deployment of the flow-restricting device 100.

Once the distal end 213 of the flexible tubular element 210 reaches a desired delivery site, the flow-restricting device 100 may be advanced distally therefrom out of the lumen 212 of the flexible tubular element 210 and into the delivery site. The inner tube 220 may be used as a pusher to advance the flow-restricting device 100 by either proximally retracting the flexible tubular element 210 relative to the inner tube 220 or by distally advancing the inner tube 220 relative to the flexible tubular element 210 or a combination of such movements. Control handles 214, 224, as described above, may be used to move the flexible tubular element 210 and inner tube 220, respectively. The flow-restricting device 100 is illustrated as advanced out of the flexible tubular element 210 in FIG. 6. If the flow-restricting device 100 is self-expanding, then the flow-restricting device 100 may commence expanding, as illustrated in FIG. 6, into an expanded deployed configuration such as illustrated in FIG. 1. If the flow-restricting device 100 is expanded by an expandable device, such as an expandable occluder 140, then the expandable device may be inflated to inflate the flow-restricting device 100. If the flow-restricting device 100 includes an occluder 140, once the flow-restricting device 100 has been advanced out of the flexible tubular element 210 and is positioned at the desired deployment site, the occluder 140 may be inflated (such as to expand the flow-restricting device 100 and/or to block or contribute to restrict passage of materials through a passage through which the flow-restricting device 100 is positioned and/or to occupy volume within the anatomical structure within which the occluder 140 is positioned).

As illustrated in FIG. 5 and FIG. 6, an inflation tube 230 may be provided for an expandable device provided either for expanding the flow-restricting device 100 and/or to remain inflated (or partially inflated) within a portion of the flow-restricting device 100 in accordance with various principles of the present disclosure as described above. The inflation tube 230 has a proximal end 231 which may be coupled to an inflation control handle 234 which may be provided on a common control handle assembly with one or both of the control handles 214, 224, or may be separately formed from either or both of the control handles 214, 224. An inflation valve or port, such as a luer lock 236, may be provided on the inflation control handle 234 for coupling with a source for an inflation medium (e.g., gas or fluid or even semi-solid inflation material).

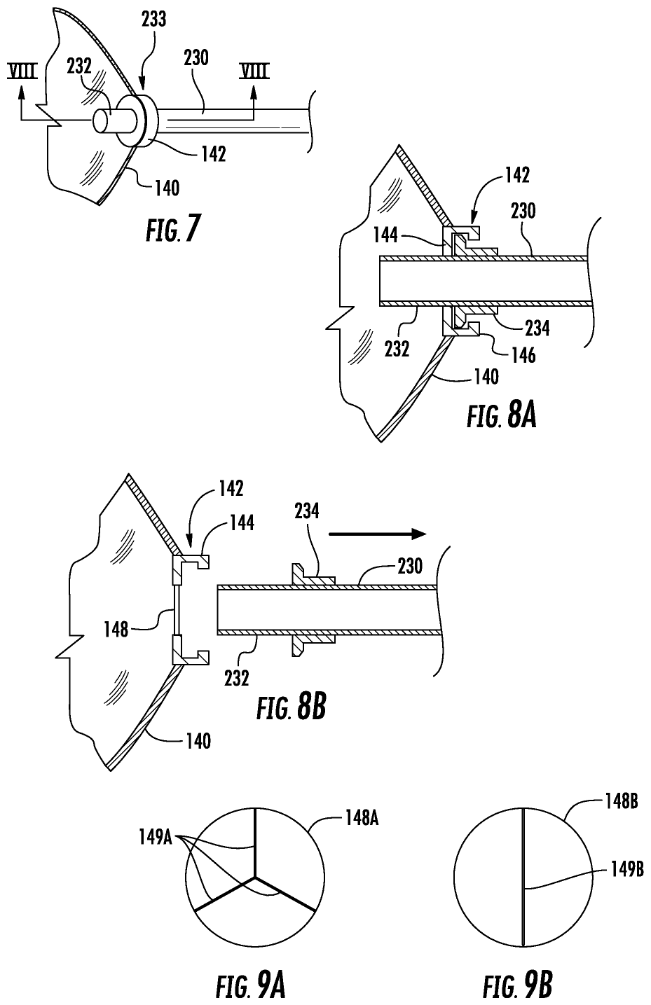

The distal end 233 of the inflation tube 230 is coupled to the occluder 140 such as via a port 142 as illustrated in the detail view of FIG. 7. The inflation tube 230 may include, at a distal end 233 thereof, an injection tube 232 configured to extend through the port 142 to facilitate passage of filling medium through the inflation tube 230 and into the occluder 140. As may be appreciated with reference to the further detailed views, along line VIII-VIII of FIG. 7, of FIG. 8A and FIG. 8B (FIG. 8B illustrating later stage of inflation than illustrated in FIG. 8A), the port 142 may include a frame or clip-receiving component 144 configured to mate with a clip insertion component 234. The clip-receiving component 144 may include holding structure 146 to hold the clip insertion component 234 in place relative to the port 142 during inflation. The clip insertion component 234 can be removed from the clip-receiving component 144 by proximally withdrawing the inflation tube 230 to disengage the clip-receiving component 144 from the clip insertion component 234, as illustrated in FIG. 8B. As may be appreciated with reference to FIG. 8B, the port 142 may include a seal 148, such as a valve seal, which selectively allows the inflation tube 230 and/or injection tube 232 access to the interior of the occluder 140 to fill the occluder 140 with inflation medium. The seal 148 may be configured to be a self-closing, one-way valve to seal the interior of the occluder 140 upon withdrawal of the inflation tube 230 and/or injection tube 232 once the occluder 140 has been inflated to the desired or medically indicated extent. In some embodiments, the seal 148 may be in the form of an elastomeric element (e.g., an elastomeric disk) extending across the opening within the port 142, and having slits therein to allow selective access therethrough. The slits may be provide in any configuration acceptable to those of ordinary skill in the art, examples of which are illustrated in FIGS. 9A and 9B. For instance, a plurality of slits 149A may be provided in a seal 148A, as illustrated in FIG. 9A, or only a single slit 149B may be provided in a seal 148B, as illustrated in FIG. 9B. Although the plurality of slits 149A are illustrated as being substantially straight and substantially equidistant from one another, other configurations are within the scope and spirit of the present disclosure.

In view of the above, it should be understood that the various embodiments illustrated in the figures have several separate and independent features, which each, at least alone, has unique benefits which are desirable for, yet not critical to, the presently disclosed flow-restricting device. Therefore, the various separate features described herein need not all be present in order to achieve at least some of the desired characteristics and/or benefits described herein. Only one of the various features may be present in a flow-restricting device formed in accordance with various principles of the present disclosure. Alternatively, one or more of the features described with reference to one embodiment can be combined with one or more of the features of any of the other embodiments provided herein. That is, any of the features described herein can be mixed and matched to create hybrid designs, and such hybrid designs are within the scope of the present disclosure. Moreover, throughout the present disclosure, reference numbers are used to indicate a generic element or feature of the disclosed embodiment. The same reference number may be used to indicate elements or features that are not identical in form, shape, structure, etc., yet which provide similar functions or benefits. Additional reference characters (such as letters, as opposed to numbers) may be used to differentiate similar elements or features from one another.

In general, it should be understood that, as described herein, an “embodiment” (such as illustrated in the accompanying Figures) may refer to an illustrative representation of an environment or article or component in which a disclosed concept or feature may be provided or embodied, or to the representation of a manner in which just the concept or feature may be provided or embodied. However such illustrated embodiments are to be understood as examples (unless otherwise stated), and other manners of embodying the described concepts or features, such as may be understood by one of ordinary skill in the art upon learning the concepts or features from the present disclosure, are within the scope of the disclosure. In addition, it will be appreciated that while the Figures may show one or more embodiments of concepts or features together in a single embodiment of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one embodiment can be used separately, or with one or more other features to yield a still further embodiment. Thus, it is intended that the present subject matter covers such modifications and variations as come within the scope of the appended claims and their equivalents.

The foregoing discussion has broad application and has been presented for purposes of illustration and description and is not intended to limit the disclosure to the form or forms disclosed herein. It will be appreciated that various aspects of the above disclosure may be applied in other passages within the body to reduce flow through such passage. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A flow-restricting device having a longitudinal extent and deployable within an anatomical structure, said flow-restricting device comprising:
a first portion;
a second portion; and
a saddle region extending between said first portion and said second portion;
wherein said first portion is not collinear with said saddle region.

2. The flow restricting device of claim 1, wherein said first portion has a noncircular cross-sectional shape at least across a region thereof configured to remain spaced apart from a selected anatomical region.

3. The flow-restricting device of claim 1, wherein:
said flow-restricting device is configured to be positioned with said saddle region extending through a passage between a first anatomical structure in which said first portion is to be positioned and a second anatomical structure in which said second portion is to be positioned;
said first portion has a first region extending along a first region of the first anatomical structure and a second region extending along a second region of the first anatomical structure; and
said first portion is angled with respect to said saddle region in a direction away from the second region of the first anatomical structure when positioned in the first anatomical structure.

4. The flow-restricting device of claim 3, wherein:
said first region of said first portion is configured to follow the contour of the first region of the first anatomical structure; and
said second region of said first portion is configured to be spaced apart from the second region of the first anatomical structure to distance said first portion from the second region of the first anatomical structure.

5. The flow-restricting device of claim 4, wherein:
said first region is convex on an outer side thereof; and
said second region is substantially straight or concave on an outer side thereof.

6. The flow-restricting device of claim 4, wherein the device cross-sectional area through said first region and said second region of said first portion is smaller than the cross-sectional area of the first anatomical structure in which the device cross-sectional area extends.

7. The flow-restricting device of claim 3, wherein:
said device is movable between a collapsed configuration and an expanded deployed configuration; and
said first portion is sized to resist migration through the passage through which said saddle region extends.

8. The flow-restricting device of claim 7, wherein:
said flow-restricting device is formed of a plurality of woven strands;
said first portion is expanded to have a greater cross-sectional dimension than said second portion; and
said first portion is formed of fewer strands than said second portion.

9. The flow-restricting device of claim 3, further comprising an occluder associated with said first portion and expandable to restrict passage of materials through the passage through which said saddle region extends.

10. The flow-restricting device of claim 3, further comprising an occluder associated with said first portion and expandable to increase the volume occupied by said first portion within the first anatomical structure.

11. The flow-restricting device of claim 1, further comprising an occluder associated with said first portion.

12. A flow-restricting device having a longitudinal extent and deployable within an anatomical structure, said flow-restricting device comprising:
a first portion;
a second portion;
a saddle region extending between said proximal portion and said distal portion; and
an occluder associated with said first portion.

13. The flow-restricting device of claim 12, wherein said occluder is selectively expandable.

14. The flow-restricting device of claim 12, wherein:
said first portion is in the form of a cage; and
said occluder is positioned within said first portion.

15. The flow-restricting device of claim 12, wherein said occluder is positioned within said first portion to restrict passage of materials towards said saddle region.

16. The flow-restricting device of claim 12, wherein said occluder is expandable to increase the volume occupied by said first portion.

17. The flow-restricting device of claim 12, wherein:
said saddle region is configured to be deployed across a pylorus;
said first portion is configured to fit within a stomach and is sized and configured to resist distal migration through the pylorus;
said second portion is configured to fit within a duodenum and is configured to resist proximal migration through the pylorus; and
said occluder is expandable within said first portion to resist distal migration of the flow-restricting device through the pylorus and to occlude passage of materials through the pylorus.

18. The flow-restricting device of claim 12, wherein:
said saddle region is configured to be deployed across a pylorus;
said first portion is configured to fit within a stomach and is sized and configured to resist distal migration through the pylorus;
said second portion is configured to fit within a duodenum and is configured to resist proximal migration through the pylorus; and
said occluder is selectively expandable within said first portion to occupy volume in the stomach to cause a feeling of satiety.

19. A system for restricting flow of material through an anatomical structure, said system comprising:
a tubular delivery device;
a flow-restricting device configured to shift between a collapsed configuration to fit within a lumen within said exterior tubular device, and an expanded deployed configuration when not positioned within said exterior tubular device;
an occluder associated with said flow-restricting device; and
an interior pusher extending through said tubular delivery device and configured to engage said flow-restricting device;
wherein movement of said interior pusher and said exterior tubular delivery device relative to each other causes deployment of said flow-restricting device from said tubular delivery device.

20. The system of claim 19, wherein said occluder is expandable, said system further comprising an inflation lumen fluidly couplable with said occluder to selectively expand said occluder.
